(19) United States
(12) Patent Application Publication
(10) Pub. No.: US 2025/0353812 A1
(43) Pub. Date: Nov. 20, 2025

(54) 2,6-DIBENZYLIDENECYCLOHEXANONE OXIME COMPOUND AND PREPARATION METHOD THEREFOR AND USE THEREOF

(71) Applicant: WENZHOU MEDICAL UNIVERSITY, Wenzhou (CN)

(72) Inventors: Guang LIANG, Wenzhou (CN); Qidong TANG, Wenzhou (CN); Wu LUO, Wenzhou (CN); Xiemin WANG, Wenzhou (CN); Wenqi WU, Wenzhou (CN); Yi WANG, Wenzhou (CN)

(73) Assignee: WENZHOU MEDICAL UNIVERSITY, Wenzhou (CN)

(21) Appl. No.: 18/871,493

(22) PCT Filed: May 30, 2023
(86) PCT No.: PCT/CN2023/097030

§ 371 Date: Dec. 4, 2024

(30) Foreign Application Priority Data
Jun. 7, 2022 (CN) 202210632102.X

(51) Int. Cl. C07C 251/44; A61K 31/15; A61K 31/341; A61K 31/351; A61K 31/357; A61K 31/40; A61K 31/4453; A61K 31/495; A61K 31/5375; A61P 1/16; C07C 251/52; C07C 251/54; C07C 251/58; C07D 295/125; C07D 307/14; C07D 309/06; C07D 317/28

(52) U.S. Cl. CPC C07C 251/44; A61K 31/15; A61K 31/341; A61K 31/351; A61K 31/357; A61K 31/40; A61K 31/4453; A61K 31/495; A61K 31/5375; A61P 1/16; C07C 251/52; C07C 251/54; C07C 251/58; C07D 295/125; C07D 307/14; C07D 309/06; C07D 317/28; C07C 2601/08; C07C 2601/14

(57) ABSTRACT

A 2,6-dibenzylidenecyclohexanone oxime compound and a preparation method and use thereof are provided. A structural fragment with anti-inflammatory activity is introduced into the 2,6-dibenzylidenecyclohexanone oxime compound, and the prepared compound has stable physicochemical properties. The 2,6-dibenzylidenecyclohexanone oxime compound has an excellent inhibitory effect on inflammatory factors and better in-vivo anti-inflammatory activity, and especially has a protective effect on hepatitis and fatty liver caused by excessive expression and release of TNF-α and/or IL-6, as well as liver inflammation and liver lipid metabolism.

### CROSS REFERENCE TO THE RELATED APPLICATIONS

This application is the national phase entry of International Application No. PCT/CN2023/097030, filed on May 30, 2023, which is based upon and claims priority to Chinese Patent Application No. 202210632102.X, filed on Jun. 7, 2022, the entire contents of which are incorporated herein by reference.

### TECHNICAL FIELD

The present invention relates to the technical field of anti-inflammatory drugs, and in particular to a 2,6-dibenzylidenecyclohexanone oxime compound and a preparation method and use thereof.

### BACKGROUND

Inflammation is an immune response of the human body itself to the environment. Generally, inflammation is beneficial for the body, but excessive inflammation can cause damage to the human body. Inflammation can be classified into acute inflammation and chronic inflammation based on the duration. Acute inflammation includes acute lung injury, sepsis, and the like, and chronic inflammation includes diabetic nephropathy, diabetic cardiomyopathy, hepatitis, fatty liver, and the like. For example, fatty liver refers to a pathological change caused by excessive accumulation of fat in liver cells. The prevalence of fatty liver is up to 25% or more at present. There is no doubt that these inflammatory diseases seriously endanger the physical and mental health of the human body, but effective clinical treatments are scarce. Therefore, the development of novel anti-inflammatory drugs remains a major challenge.

Hepatitis is a general term for inflammation of the liver. Generally, hepatitis refers to the destruction of liver cells and liver function caused by a variety of pathogenic factors such as viruses, bacteria, parasites, chemical poisons, drugs, alcohol, and autoimmune factors, which causes a series of physical discomfort symptoms and abnormal liver function indicators. Fatty liver is a pathological condition caused by excessive accumulation of fat in the liver due to the combined action of multiple factors. Abnormal synthesis and secretion functions of hepatitis-related liver cells are an important cause of fatty liver. Inflammatory damage may be related to the activation of inflammatory signaling pathways MAPKs and NF-κB, which can be activated by free fatty acids (such as palmitic acid) and microbial products (such as LPS), leading to the release of proinflammatory molecules such as tumornecrosis factor alpha (TNF-α), interleukin-1beta (IL-1β), and interleukin-6 (IL-6). Excessive inflammation not only leads to hepatocyte damage and fibrosis, but also promotes liver lipid accumulation. Blocking these signaling pathways with drug inhibitors or gene knockout can alleviate the progression of inflammatory fatty liver, further confirming that inflammatory damage in liver is mainly attributed to the activation of inflammatory signaling pathways.

Therefore, how to provide a novel compound having an excellent anti-inflammatory effect is of great significance to human health.

### SUMMARY

An objective of the present invention is to provide a 2,6-dibenzylidenecyclohexanone oxime compound, and a preparation method and use thereof, wherein the compound has a significant anti-inflammatory activity and can be used to prepare a medicament for treating hepatitis and fatty liver.

To achieve the above objective, the present invention provides the following technical solutions.

The present invention provides a 2,6-dibenzylidenecyclohexanone oxime compound, wherein the compound is a compound with a structure of formula I or a pharmaceutically acceptable salt thereof:

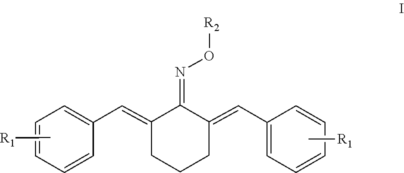

in the formula I, R₁ is independently one of fluorine, chlorine, trifluoromethyl, methoxy, or hydroxyl; and

R₂ is independently one of hydrogen,

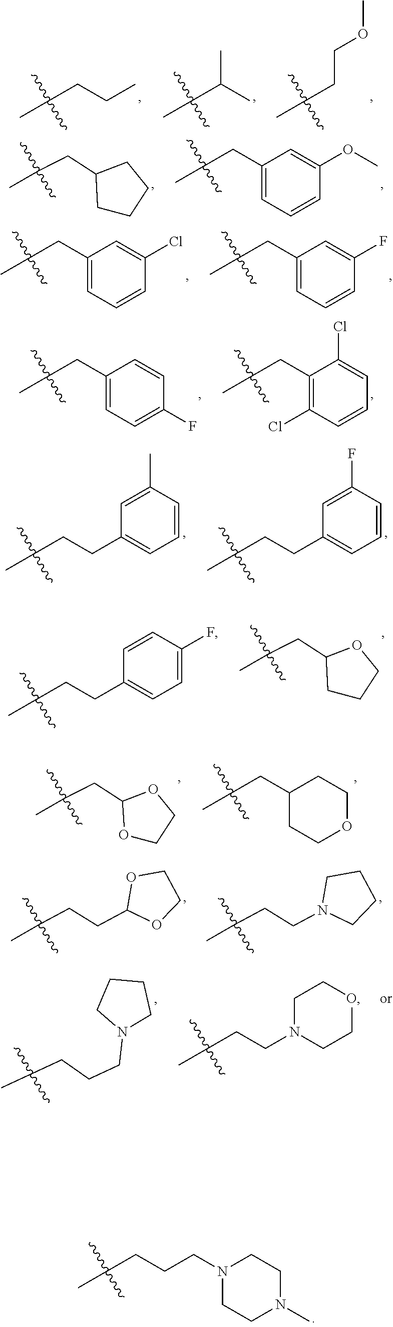

Preferably, the 2,6-dibenzylidenecyclohexanone oxime compound is the following compound or a pharmaceutically acceptable salt thereof:
2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(pyrrolidin-1-yl)ethyl oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one isopropyl oxime;
2,6-(E,E)-(2-(trifluoromethyl)benzylidene)cyclohexanone oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one-((tetrahydrofuran-2-yl) methyl)oxime;
2,6-(E,E)-(2-(trifluoromethyl)benzylidene)cyclohexan-1-one- ((1,3-dioxolan-2-yl) methyl)oxime;
2,6-(E,E)-(2-(trifluoromethyl)benzylidene)cyclohexan-1-one-(tetrahydro-2H-pyran-4-yl) methyloxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(fluoro)benzylidene)cyclohexan-1-one isopropyl oxime;
2,6-(E,E)-(4-(methoxy)benzylidene)cyclohexan-1-one-(2-methoxyethyl)oxime;
2,6-(E,E)-(4-(hydroxy)benzylidene)cyclohexan-1-one cyclopentylmethyl oxime; and
2,6-(E,E)-(3,4-(dichloro)benzylidene)cyclohexan-1-one-2-(morpholinoethyl)oxime.

The present invention further provides use of a 2,6-dibenzylidenecyclohexanone oxime compound in preparing a medicament for the treatment of inflammation.

The present invention further provides use of a 2,6-dibenzylidenecyclohexanone oxime compound in preparing a medicament for the treatment of fatty liver, wherein the 2,6-dibenzylidenecyclohexanone oxime compound is 2,6-(E,E)-(2-(trifluoromethyl) benzylidene)cyclohexan-1-one propyl oxime or 2,6-(E,E)-(2-(trifluoromethyl) benzylidene)cyclohexanone oxime.

The present invention further provides a pharmaceutical preparation, which includes an active ingredient and a pharmaceutic adjuvant; wherein the active ingredient includes the 2,6-dibenzylidenecyclohexanone oxime compound according to claim 1 or 2; and the pharmaceutical preparation is one of an injection, a tablet, a capsule, an aerosol, a suppository, a film, a dropping pill, an ointment, a controlled-release agent, a sustained-release agent, or a nano-preparation.

It can be known from the technical solutions that, compared with the prior art, the present invention has the following beneficial effects.

The present invention introduces a structural fragment with anti-inflammatory activity into a compound, and the prepared compound has stable physicochemical properties. Experiments have shown that the 2,6-dibenzylidenecyclohexanone oxime compound has an excellent inhibitory effect on inflammatory factors and better in-vivo anti-inflammatory activity, and especially has a protective effect on hepatitis and fatty liver caused by excessive expression and release of TNF-α and/or IL-6.

### BRIEF DESCRIPTION OF THE DRAWINGS

To more clearly illustrate the technical solutions in the examples of the present invention or in the prior art, the drawings used in the description of the examples or the prior art are briefly introduced below.

FIG. 1 is a graph showing the dose-effect relationship of compounds of Examples in inhibiting the release of IL-6 from J774A.1 cells stimulated by LPS;

FIG. 2 is a graph showing the dose-effect relationship of compounds of Examples in inhibiting the release of TNF-α from J774A. 1 cells stimulated by LPS;

FIGS. 3A-3D are graphs showing the protective effect of the compound of Example 2 on liver inflammation in obese mice;

where FIG. 3A is F4/80 immunohistochemical staining pattern; FIG. 3B is the mRNA level of tumor necrosis factor (TNF-α) in liver tissue; FIG. 3C is the mRNA level of interleukin 6 (IL-6) in liver tissue; FIG. 3D is the mRNA level of interleukin 1β (IL-1β) in liver tissue;

FIGS. 4A-4E are graphs showing the protective effect of the compound of Example 9 on liver inflammation in obese mice;

where FIG. 4A is F4/80 immunohistochemical staining pattern; FIG. 4B is the mRNA level of tumor necrosis factor (TNF-α) in liver tissue; FIG. 4C is the mRNA level of interleukin 6 (IL-6) in liver tissue; FIG. 4D is the mRNA level of interleukin 1β(IL-1β) in liver tissue; and FIG. 4E is a Western blot analysis and quantitation plot of IKB-α and GAPDH protein levels in liver tissue;

FIGS. 5A-5F are graphs showing the protective effect of the compound of Example 2 on liver lipid metabolism in obese mice;

where FIG. 5A is a graph of mouse liver tissue morphology assessed by hematoxylin-eosin staining; FIG. 5B is a graph of lipid accumulation in mouse liver assessed by oil red staining; FIG. 5C is a statistical graph of oil red staining; FIG. 5D is the mRNA level of Acaca in mouse liver tissue; FIG. 5E is the mRNA level of Srebpl in mouse liver tissue; and FIG. 5F is the mRNA level of Ppar-α in mouse liver tissue;

FIGS. 6A-6F are graphs showing the protective effect of the compound of Example 9 on liver lipid metabolism in obese mice;

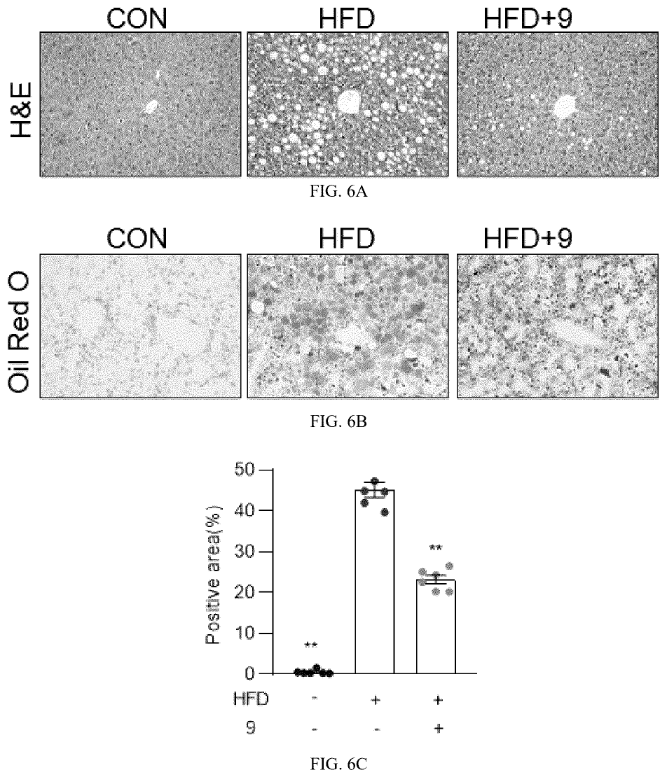

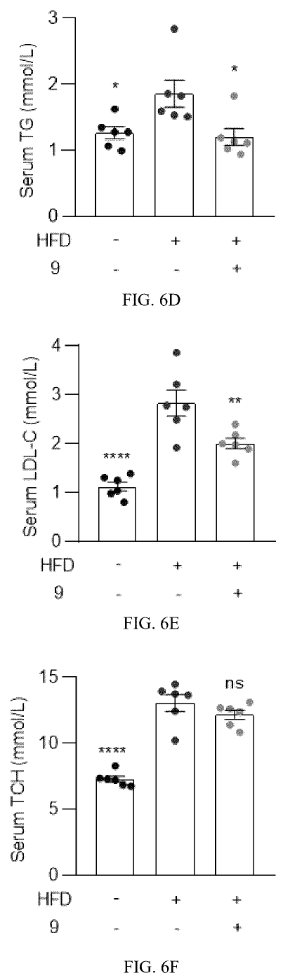

where FIG. 6A is a graph of mouse liver tissue morphology assessed by hematoxylin-eosin staining; FIG. 6B is a graph of lipid accumulation in mouse liver assessed by oil red staining; FIG. 6C is a statistical graph of oil red staining; FIG. 6D is a statistical graph of serum triglycerides (serum TG); FIG. 6E is a statistical graph of serum low-density lipoprotein (serum LDL-C); and FIG. 6F is a statistical graph of serum total cholesterol (serum TCH).

### DETAILED DESCRIPTION OF THE EMBODIMENTS

The present invention provides a 2,6-dibenzylidenecyclohexanone oxime compound, wherein the compound is a compound with a structure of formula I or a pharmaceutically acceptable salt, a hydrate, a solvate or a prodrug thereof:

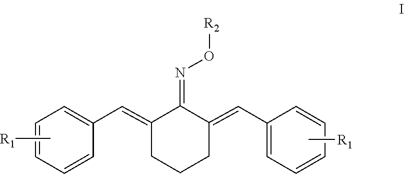

in the formula I, R₁ is independently one of hydrogen, halogen, halogenated alkyl, alkoxy or hydroxyl; R₂ is hydrogen or a flexible fragment;

the flexible fragment has the following structure:

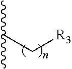

wherein n is 0 to 3, and R₃ is independently one of alkyl, alkoxy, cycloalkyl, dialkylamino, 5- to 6-membered N-containing heterocyclic group, a benzene ring, or a substituted benzene ring. In the present invention, in the formula I, R₁ is preferably one of hydrogen, fluorine, chlorine, bromine, trifluoromethyl, methoxy, or hydroxyl independently; and

R₂ is preferably hydrogen, ethyl,

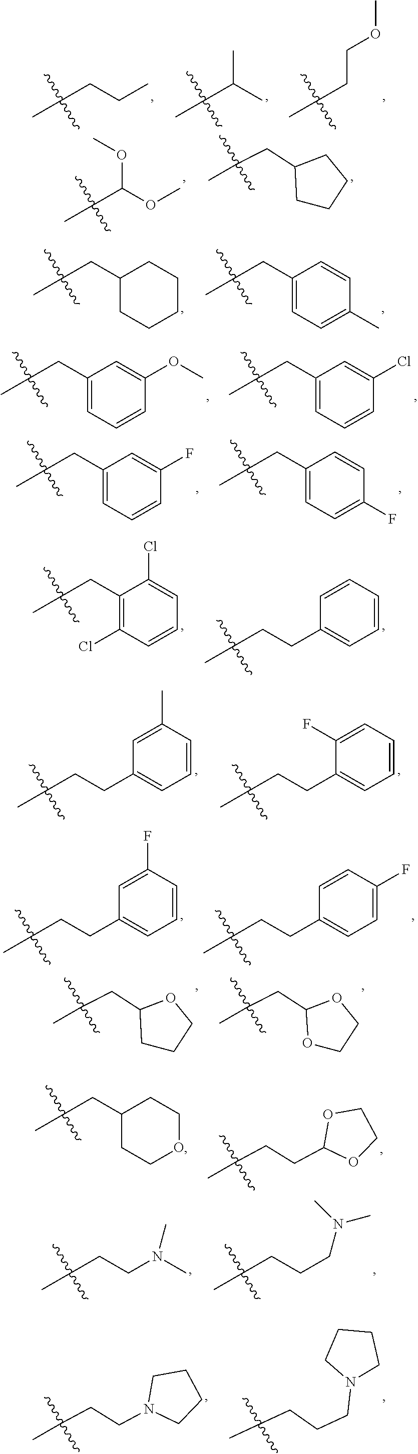

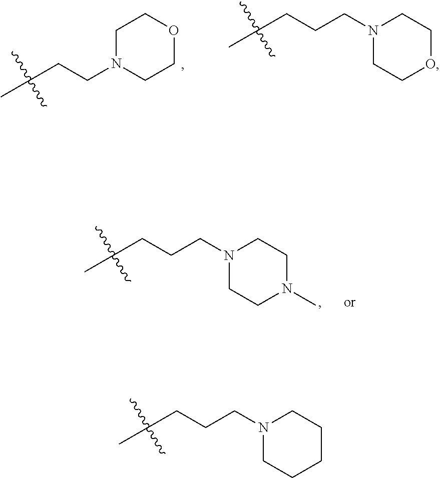 [SMILES: *CCN1CCOCC1 ; *CCCN1CCOCC1 ; *CCCN1CCN(C)CC1 ; *CCCN1CCCCC1]

independently.

In the present invention, the 2,6-dibenzylidenecyclohexanone oxime compound is preferably the following compound or a pharmaceutically acceptable salt, a hydrate, a solvate or a prodrug thereof:
2,6-(E,E)-(2-(trifluoromethyl)benzylidene)cyclohexan-1-one ethyl oxime;
2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(dimethylamino)ethyl oxime;
2,6-(E,E)-(2-(trifluoromethyl)benzylidene)cyclohexan-1-one-3-(dimethylamino)propyl oxime;
2,6-(E,E)-(2-(trifluoromethyl)benzylidene)cyclohexan-1-one-2-(pyrrolidin-1-yl)ethyl)oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one-3-(morpholinopropyl)oxime;
2,6-(E,E)-(2-(trifluoromethyl)benzylidene)cyclohexanone oxime;
2,6-(E,E)-(2-(trifluoromethyl)benzylidene)cyclohexan-1-one isopropyl oxime;
2,6-(E,E)-(2-(trifluoromethyl)benzylidene)cyclohexan-1-one-(2-methoxyethyl)oxime;
2,6-(E,E)-(2-(trifluoromethyl)benzylidene)cyclohexan-1-one-(2,2-dimethoxyethyl)oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one cyclohexylmethyl oxime;
2,6-(E,E)-(2-(trifluoromethyl)benzylidene)cyclohexan-1-one-4-(methylbenzyl)oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-ketophenethyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-2-(fluorophenethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one- ((tetrahydrofuran-2-yl) methyl)oxime;
2,6-(E,E)-(2-(trifluoromethyl)benzylidene)cyclohexan-1-one- ((1,3-dioxolan-2-yl) methyl)oxime;
2,6-(E,E)-(2-(trifluoromethyl)benzylidene)cyclohexan-1-one-(tetrahydro-2H-pyran-4-yl) methyl oxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(trifluoromethyl)benzylidene)cyclohexan-1-one-(3-(piperidin-1-yl)propyl) oxime;
2,6-(E,E)-(benzylidene)cyclohexan-1-one propyl oxime;
2,6-(E,E)-(2-(fluoro)benzylidene)cyclohexan-1-one isopropyl oxime;
2,6-(E,E)-(4-(bromo)benzylidene)cyclohexan-1-one isopropyl oxime;
2,6-(E,E)-(4-(methoxy)benzylidene)cyclohexan-1-one-(2-methoxyethyl)oxime;
2,6-(E,E)-(4-(methoxy)benzylidene)cyclohexan-1-one cyclopentylmethyl oxime;
2,6-(E,E)-(4-(hydroxy)benzylidene)cyclohexan-1-one cyclopentylmethyl oxime;
2,6-(E,E)-(4-(hydroxy)benzylidene)cyclohexan-1-one-2-(morpholinoethyl)oxime;
2,6-(E,E)-(3,4-(dichloro)benzylidene)cyclohexan-1-one-2-(morpholinoethyl)oxime; and
2,6-(E,E)-(2-(hydroxy)benzylidene)cyclohexan-1-one propyl oxime.

In the present invention, the 2,6-dibenzylidenecyclohexanone oxime compound can generate a pharmaceutically acceptable salt with acid based on the conventional method in the field; the acid preferably includes an inorganic acid or an organic acid, and further preferably hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, trifluoroacetic acid, maleic acid, citric acid, fumaric acid, tartaric acid, benzenesulfonic acid, benzoic acid, or p-toluenesulfonic acid.

In the present invention, the compound further includes a prodrug of the 2,6-dibenzylidenecyclohexanone oxime; and the prodrug is preferably a derivative of 2,6-dibenzylidenecyclohexanone oxime compound. The prodrug itself has weak or even no activity, but is converted to a corresponding biologically active form under physiological conditions (e.g., by metabolism, solvolysis, or otherwise) upon administration.

The present invention further provides preparation method for a 2,6-dibenzylidenecyclohexanone oxime compound, which includes the following steps:

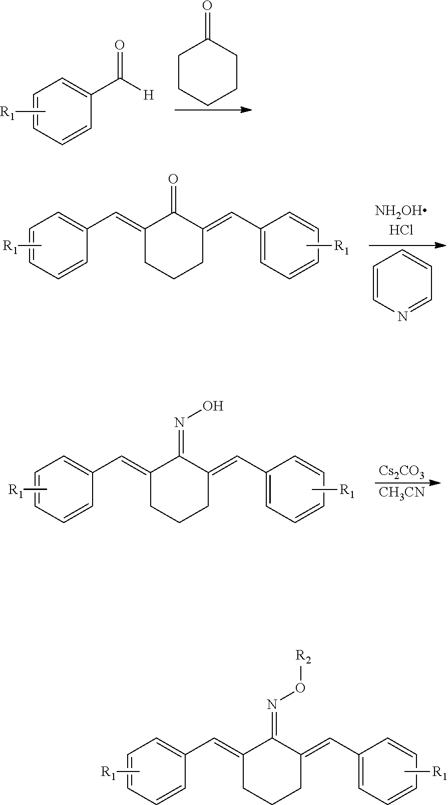

(1) mixing an aldehyde compound, cyclohexanone, an alkali solution and a solvent, and reacting at room temperature to obtain a product 1;

(2) mixing the product 1, hydroxylamine hydrochloride, pyridine and a solvent for reflux reaction to obtain a product 2; and

(3) mixing the product 2, bromide, cesium carbonate and a solvent for reaction to obtain the 2,6-dibenzylidenecyclohexanone oxime compound.

In the present invention, a molar-to-volume ratio of the aldehyde compound, the cyclohexanone, the alkali solution and the solvent in the step (1) is preferably 0.4-0.7 mmol: 0.3-1 mmol: 0.03-0.09 mL: 1-10 mL, further preferably 0.47-0.62 mmol: 0.4-0.9 mmol: 0.04-0.08 mL: 2-8 mL, and more preferably 0.57 mmol: 0.6 mmol: 0.06 mL: 5 mL; the reaction is performed for preferably 8-15 h, further preferably 9-13 h, and more preferably 11 h; the aldehyde compound is preferably benzaldehyde containing R₁; the alkali solution is preferably a sodium hydroxide solution, further preferably a sodium hydroxide solution with a mass concentration of 20-40%, and more preferably a sodium hydroxide solution with a mass concentration of 30%; and the solvent is preferably absolute ethanol.

In the present invention, a molar-to-volume ratio of the product 1, the hydroxylamine hydrochloride, the pyridine and the solvent in the step (2) is preferably 0.1-0.5 mmol: 0.3-0.7 mmol: 0.3-0.7 mmol: 1-10 mL, further preferably 0.17-0.42 mmol: 0.4-0.6 mmol: 0.34-0.65 mmol: 3-9 mL, and more preferably 0.27 mmol: 0.5 mmol: 0.43 mmol: 8 mL; the reaction is performed at a temperature of preferably 70-90° C., further preferably 74-86° C., and more preferably 82° C.; the reaction is performed for preferably 1-5 h, further preferably 2-5 h, and more preferably 4 h; and the solvent is preferably absolute ethanol.

In the present invention, a molar-to-volume ratio of the product 2, the bromide, the cesium carbonate and the solvent in the step (3) is preferably 0.1-0.4 mmol: 0.2-0.6 mmol: 0.4-1.5 mmol: 1-10 mL, further preferably 0.12-0.33 mmol: 0.3-0.5 mmol: 0.6-1.3 mmol: 3-7 mL, and more preferably 0.24 mmol: 0.4 mmol: 0.9 mmol: 6 mL; the reaction is performed at a temperature of preferably 70-90° C., further preferably 72-87° C., and more preferably 78° C.; the reaction is performed for preferably 1-5 h, further preferably 1-4 h, and more preferably 3 h; the bromide is preferably a bromine-containing compound containing R₂; and the solvent is preferably acetonitrile.

In the present invention, after the reactions in the steps (1) to (3) are completed, post-treatment is further included; the post-treatment specifically includes: after the reactions are completed, removing the solvent, and sequentially extracting, washing, drying and separating the product by column chromatography. The method of extraction, washing, drying and column chromatography separation is not limited in the present invention, and may be any method known to those skilled in the art.

In the present invention, the starting materials used are all prepared by the methods described in these formulae or by methods well known to those skilled in the art, or are commercially available. All final compounds of the present invention are prepared by the methods described in these formulae or by methods analogous thereto, which are well known to those skilled in the art. All of the variable factors applied in these formulae are as defined below or as defined above.

The present invention further provides use of a 2,6-dibenzylidenecyclohexanone oxime compound in preparing a medicament for the treatment of inflammation or inflammation-related diseases.

In the present invention, the medicament for the treatment of inflammation or inflammation-related diseases treats inflammation or inflammation-related diseases by inhibiting the release of inflammatory cytokines.

In the present invention, the inflammation or inflammation-related diseases include sepsis, acute lung injury, arthritis, colorectalitis, hepatitis caused by various factors, fatty liver, or chronic diseases in which chronic inflammation is an important pathological pathway; the chronic disease includes diabetic complications, atherosclerosis, obesity complications or hypertension complications; the diabetic complications include diabetic nephropathy or diabetic cardiomyopathy.

The present invention further provides a pharmaceutical preparation, which includes an active ingredient and a pharmaceutic adjuvant; and the active ingredient includes the 2,6-dibenzylidenecyclohexanone oxime compound. The 2,6-dibenzylidenecyclohexanone oxime compound and the pharmaceutically acceptable salt, the hydrate or the solvate thereof are used as active ingredients and mixed with a pharmaceutically acceptable pharmaceutic adjuvant to prepare a pharmaceutical preparation. The compound of the present invention may also be used in combination with other active ingredients as long as other adverse effects such as allergic reactions do not occur. The pharmaceutical preparation may be administered orally or parenterally (e.g., intravenously, subcutaneously, intraperitoneally, or topically), and if certain drugs are unstable under gastric conditions, they may be formulated as enteric-coated tablets.

In the present invention, the pharmaceutically acceptable pharmaceutic adjuvant includes a carrier or an excipient.

In the present invention, the carrier includes one or more of a binder, a lubricant, a disintegrating agent, a cosolvent, a diluent, a stabilizing agent, a suspending agent, a pigment-free substance, a flavoring agent, a preservative, a solubilizer, and a matrix.

In the present invention, the excipient includes any diluent or adjuvant which can be used in the pharmaceutical field.

In the present invention, the pharmaceutical preparation is one of an injection, a tablet, a capsule, an aerosol, a suppository, a film, a dropping pill, an ointment, a controlled-release agent, a sustained-release agent, or a nano-preparation.

The technical solutions in the examples of the present invention will be clearly and completely described below. Apparently, the described examples are merely a part, rather than all of the examples of the present invention. Based on the examples of the present invention, all other examples obtained by those of ordinary skill in the art without creative efforts shall fall within the protection scope of the present invention.

According to the preparation method of the formula I, the compounds of Examples 1 to 40 are prepared, and the structural formulas are shown in Table 1.

TABLE 1
Structural formulas of compounds of Examples 1 to 40

| No. | Structural formula |
|---|---|
| 1 | 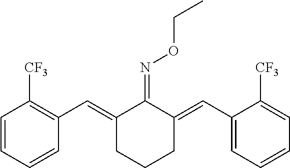 [SMILES: CCON=C1/C(=C/c2ccccc2C(F)(F)F)CCC/C1=C\c1ccccc1C(F)(F)F] |
| 2 | 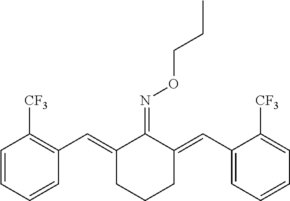 [SMILES: CCCON=C1/C(=C/c2ccccc2C(F)(F)F)CCC/C1=C\c1ccccc1C(F)(F)F] |
| 3 | 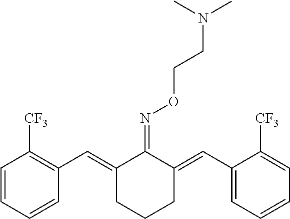 [SMILES: CN(C)CCON=C1/C(=C/c2ccccc2C(F)(F)F)CCC/C1=C\c1ccccc1C(F)(F)F] |
| 4 | 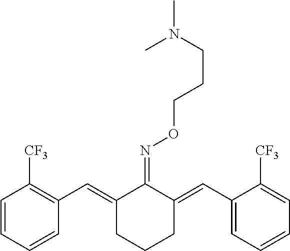 [SMILES: CN(C)CCCON=C1/C(=C/c2ccccc2C(F)(F)F)CCC/C1=C\c1ccccc1C(F)(F)F] |
| 5 | 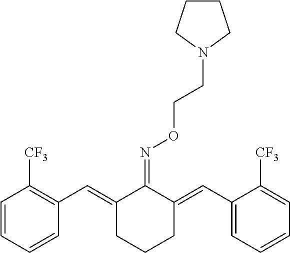 [SMILES: FC(F)(F)c1ccccc1/C=C1\CCC/C(=C\c2ccccc2C(F)(F)F)C1=NOCCN1CCCC1] |
| 6 | 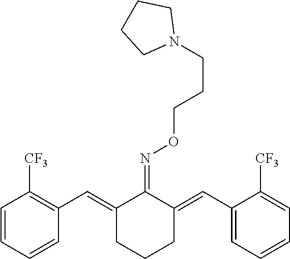 [SMILES: FC(F)(F)c1ccccc1/C=C1\CCC/C(=C\c2ccccc2C(F)(F)F)C1=NOCCCN1CCCC1] |
| 7 | 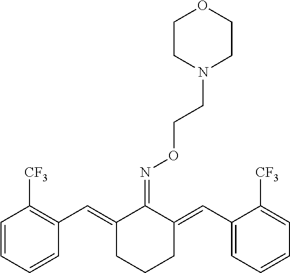 [SMILES: FC(F)(F)c1ccccc1/C=C1\CCC/C(=C\c2ccccc2C(F)(F)F)C1=NOCCN1CCOCC1] |
| 8 | 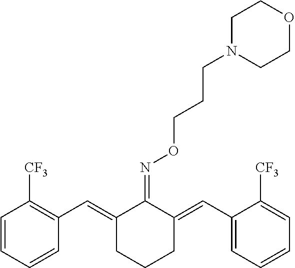 [SMILES: FC(F)(F)c1ccccc1/C=C1\CCC/C(=C\c2ccccc2C(F)(F)F)C1=NOCCCN1CCOCC1] |
| 9 | 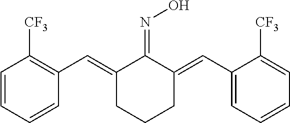 [SMILES: ON=C1/C(=C/c2ccccc2C(F)(F)F)CCC/C1=C\c1ccccc1C(F)(F)F] |
| 10 | 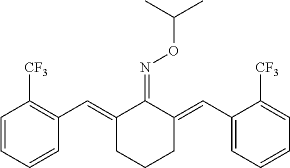 [SMILES: CC(C)ON=C1/C(=C/c2ccccc2C(F)(F)F)CCC/C1=C\c1ccccc1C(F)(F)F] |
| 11 | 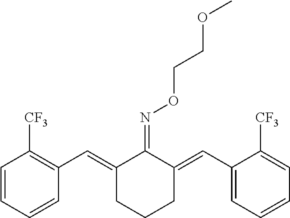 [SMILES: COCCON=C1/C(=C/c2ccccc2C(F)(F)F)CCC/C1=C\c1ccccc1C(F)(F)F] |
| 12 | 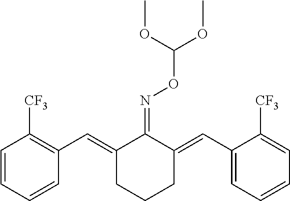 [SMILES: COC(OC)ON=C1/C(=C/c2ccccc2C(F)(F)F)CCC/C1=C\c1ccccc1C(F)(F)F] |
| 13 | 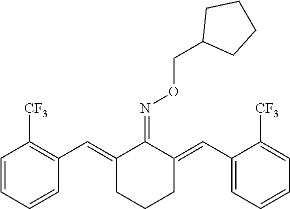 [SMILES: FC(F)(F)c1ccccc1/C=C1\CCC/C(=C\c2ccccc2C(F)(F)F)C1=NOCC1CCCC1] |
| 14 | 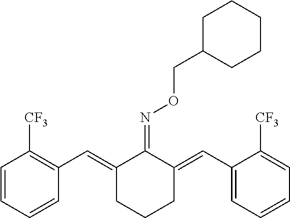 [SMILES: FC(F)(F)c1ccccc1/C=C1\CCC/C(=C\c2ccccc2C(F)(F)F)C1=NOCC1CCCCC1] |
| 15 | 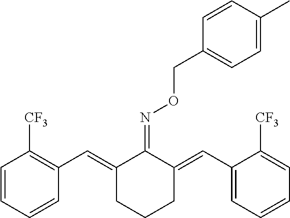 [SMILES: Cc1ccc(CON=C2/C(=C/c3ccccc3C(F)(F)F)CCC/C2=C\c2ccccc2C(F)(F)F)cc1] |
| 16 | 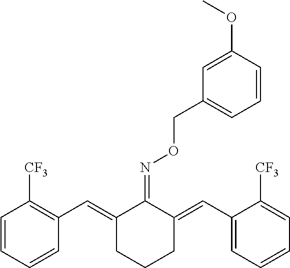 [SMILES: COc1cccc(CON=C2/C(=C/c3ccccc3C(F)(F)F)CCC/C2=C\c2ccccc2C(F)(F)F)c1] |
| 17 | 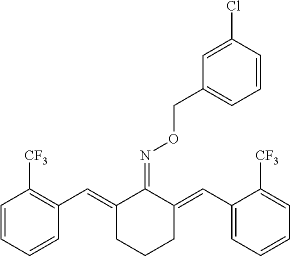 [SMILES: FC(F)(F)c1ccccc1/C=C1\CCC/C(=C\c2ccccc2C(F)(F)F)C1=NOCc1cccc(Cl)c1] |
| 18 | 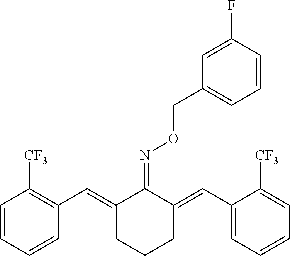 [SMILES: Fc1cccc(CON=C2/C(=C/c3ccccc3C(F)(F)F)CCC/C2=C\c2ccccc2C(F)(F)F)c1] |
| 19 | 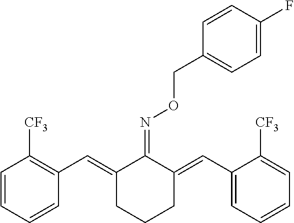 [SMILES: Fc1ccc(CON=C2/C(=C/c3ccccc3C(F)(F)F)CCC/C2=C\c2ccccc2C(F)(F)F)cc1] |
| 20 | 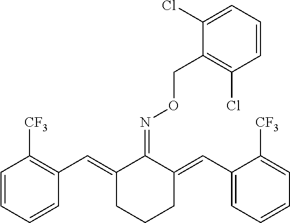 [SMILES: FC(F)(F)c1ccccc1/C=C1\CCC/C(=C\c2ccccc2C(F)(F)F)C1=NOCc1c(Cl)cccc1Cl] |
| 21 | 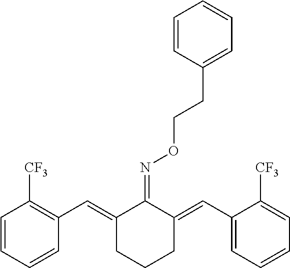 [SMILES: FC(F)(F)c1ccccc1/C=C1\CCC/C(=C\c2ccccc2C(F)(F)F)C1=NOCCc1ccccc1] |
| 22 | 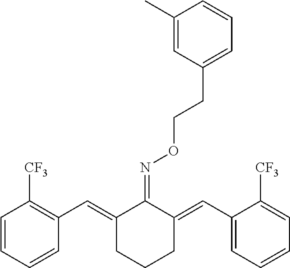 [SMILES: Cc1cccc(CCON=C2/C(=C/c3ccccc3C(F)(F)F)CCC/C2=C\c2ccccc2C(F)(F)F)c1] |
| 23 | 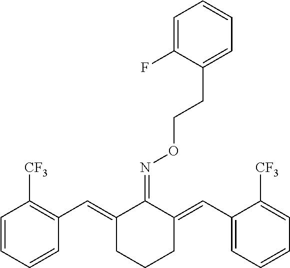 [SMILES: Fc1ccccc1CCON=C1/C(=C/c2ccccc2C(F)(F)F)CCC/C1=C\c1ccccc1C(F)(F)F] |
| 24 | 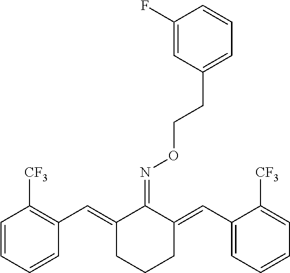 [SMILES: Fc1cccc(CCON=C2/C(=C/c3ccccc3C(F)(F)F)CCC/C2=C\c2ccccc2C(F)(F)F)c1] |
| 25 | 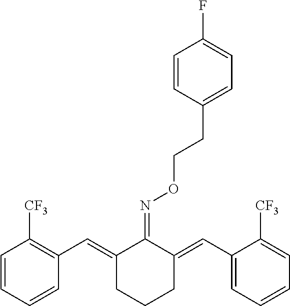 [SMILES: Fc1ccc(CCON=C2/C(=C/c3ccccc3C(F)(F)F)CCC/C2=C\c2ccccc2C(F)(F)F)cc1] |
| 26 | 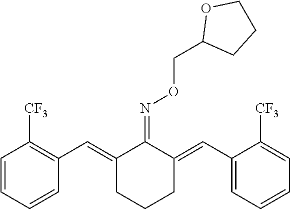 [SMILES: FC(F)(F)c1ccccc1/C=C1\CCC/C(=C\c2ccccc2C(F)(F)F)C1=NOCC1CCCO1] |
| 27 | 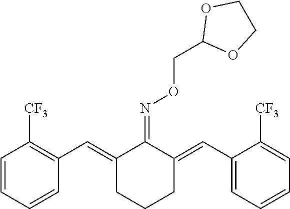 [SMILES: FC(F)(F)c1ccccc1/C=C1\CCC/C(=C\c2ccccc2C(F)(F)F)C1=NOCC1OCCO1] |
| 28 | 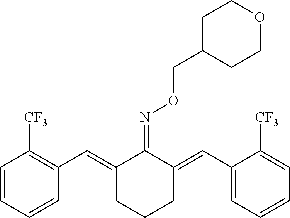 [SMILES: FC(F)(F)c1ccccc1/C=C1\CCC/C(=C\c2ccccc2C(F)(F)F)C1=NOCC1CCOCC1] |
| 29 | 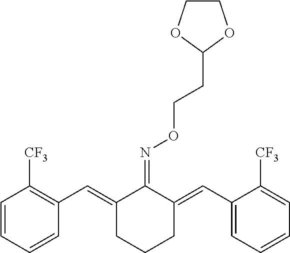 [SMILES: FC(F)(F)c1ccccc1/C=C1\CCC/C(=C\c2ccccc2C(F)(F)F)C1=NOCCC1OCCO1] |
| 30 | 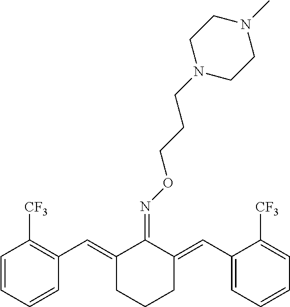 [SMILES: CN1CCN(CCCON=C2/C(=C/c3ccccc3C(F)(F)F)CCC/C2=C\c2ccccc2C(F)(F)F)CC1] |
| 31 | 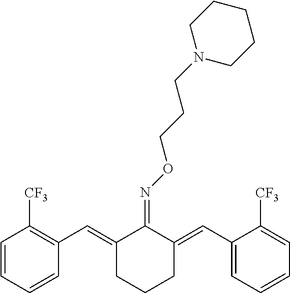 [SMILES: FC(F)(F)c1ccccc1/C=C1\CCC/C(=C\c2ccccc2C(F)(F)F)C1=NOCCCN1CCCCC1] |
| 32 | 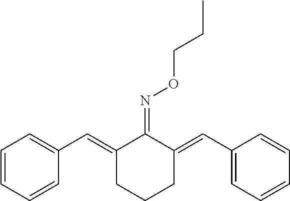 [SMILES: CCCON=C1/C(=C/c2ccccc2)CCC/C1=C\c1ccccc1] |
| 33 | 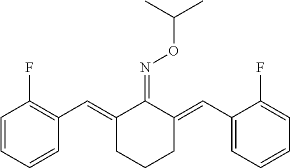 [SMILES: CC(C)ON=C1/C(=C/c2ccccc2F)CCC/C1=C\c1ccccc1F] |
| 34 | 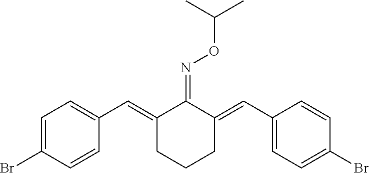 [SMILES: CC(C)ON=C1/C(=C/c2ccc(Br)cc2)CCC/C1=C\c1ccc(Br)cc1] |
| 35 | 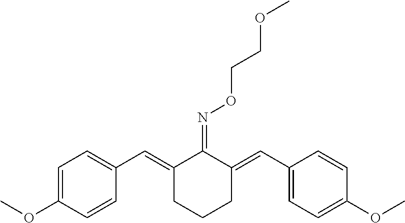 |
| 36 | 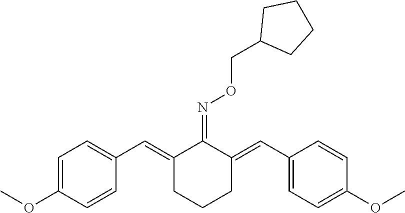 |
| 37 | 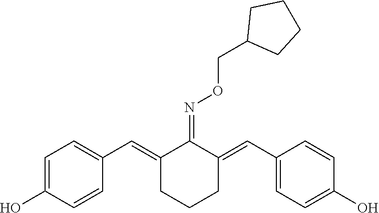 [SMILES: Oc1ccc(/C=C2\CCC/C(=C\c3ccc(O)cc3)C2=NOCC2CCCC2)cc1] |
| 38 | 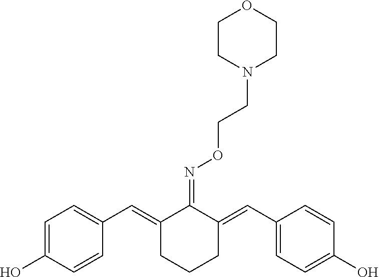 [SMILES: Oc1ccc(/C=C2\CCC/C(=C\c3ccc(O)cc3)C2=NOCCN2CCOCC2)cc1] |
| 39 | 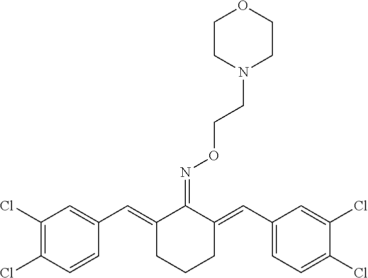 [SMILES: Clc1ccc(/C=C2\CCC/C(=C\c3ccc(Cl)c(Cl)c3)C2=NOCCN2CCOCC2)cc1Cl] |
| 40 | 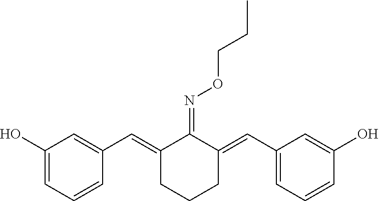 [SMILES: CCCON=C1/C(=C/c2cccc(O)c2)CCC/C1=C\c1cccc(O)c1] |

### Example 1

2,6-(E,E)-(2-(trifluoromethyl)benzylidene)cyclohexan-1-one ethyl oxime

### (1) Preparation of 2,6-(E,E)-(2-(trifluoromethyl)benzylidene)cyclohexan-1-one

In a 25 mL round-bottomed flask, o-trifluoromethylbenzaldehyde (100 mg,0.57 mmol), cyclohexanone (28 mg, 0.45 mmol) and a 40% NaOH solution (57.5 μL) were sequentially added to absolute ethanol (5 mL) for reaction at room temperature for 10 h. The ethanol was evaporated under reduced pressure and extracted with EA, and the organic layer was washed three times with saturated sodium chloride solution and dried over anhydrous magnesium sulfate. The EA was evaporated under reduced pressure, and the residue was separated by column chromatograph (PE:EA=4:1) to obtain the 2,6-(E,E)-(2-(trifluoromethyl) benzylidene)cyclohexan-1-one with a yield of 80%.

(2) Preparation of 2,6-(E,E)-(2-(trifluoromethyl)benzylidene)cyclohexanone oxime

In a 25 mL round-bottom flask, the 2,6-(E,E)-(2-(trifluoromethyl)benzylidene)cyclohexan-1-one (100 mg, 0.24 mmol) synthesized in step I, hydroxylamine hydrochloride (33 mg, 0.48 mmol) and pyridine (38 mg, 0.48 mmol) were added to anhydrous ethanol (5 mL) in sequence and refluxed at 80° C. for 4 h. After the reaction was completed, the ethanol was evaporated under reduced pressure and extracted with EA, and the organic layer was washed three times with saturated sodium chloride solution and dried over anhydrous magnesium sulfate. The EA was evaporated under reduced pressure, and the residue was separated by column chromatograph (PE:EA=10:1) to obtain the 2,6-(E,E)-(2-(trifluoromethyl)benzylidene)cyclohexanone oxime with a yield of 85%.

(3) Preparation of 2,6-(E,E)-(2-(trifluoromethyl)benzylidene)cyclohexan-1-one ethyl oxime

In a 25 mL round-bottom flask, the 2,6-(E,E)-(2-(trifluoromethyl) benzylidene)cyclohexanone oxime (100 mg, 0.25 mmol) synthesized in step II, bromoethane (41 mg, 0.375 mmol) and cesium carbonate (163 mg, 0.5 mmol) were added to 5 mL of acetonitrile in sequence and reacted at 80° C. for 4 h. After the reaction was completed, acetonitrile was evaporated to dryness and extracted with EA, the organic layer was washed three times with saturated sodium chloride solution and dried over anhydrous magnesium sulfate, the EA was evaporated under reduced pressure, and the residue was separated by column chromatograph (PE:EA=30:1) to obtain the 2,6-(E,E)-(2-(trifluoromethyl) benzylidene)cyclohexan-1-one ethyl oxime with a yield of 60%.

ESI-MS m/z: 454.20. ¹H NMR (500 MHZ, CDCl₃) δ 7.60 (dd, J=7.7, 4.2 Hz, 2H), 7.42 (q, J=8.0 Hz, 3H), 7.28 (dt, J=14.0, 7.0 Hz, 4H), 6.98 (s, 1H), 4.19 (q, J=7.1 Hz, 2H), 2.41 (t, J =5.8 Hz, 2H), 2.31-2.26 (m, 2H), 1.50 (dd, J=12.6, 6.2 Hz, 2H), 1.27 (t, J=7.1 Hz, 3H).

According to the method of Example 1, the compounds of Examples 2 to 40 were prepared.

### Example 2

2,6-(E,E)-(2-(trifluoromethyl)benzylidene)cyclohexan-1-one propyl oxime

### ESI-MS m/z: 468.00.

### Example 3

2,6-(E,E)-(2-(trifluoromethyl)benzylidene)cyclohexan-1-one-2-(dimethylamino)ethyl oxime

### ESI-MS m/z: 497.20. 1H NMR (400 MHz, CDCl₃) δ 7.67 (d, J=7.6 Hz, 2H), 7.54-7.41 (m, 3H), 7.40-7.28 (m, 4H), 7.05 (s, 1H), 4.36 (t, J=5.5 Hz, 2H), 2.76 (t, J=5.5 Hz, 2H), 2.47 (t, J=5.7 Hz, 2H), 2.35 (s, 8H), 1.61-1.51 (m, 2H).

### Example 4

2,6-(E,E)-(2-(trifluoromethyl)benzylidene)cyclohexan-1-one-3-(dimethylamino)propyl oxime

### ESI-MS m/z: 511.20.

### Example 5

2,6-(E,E)-(2-(trifluoromethyl)benzylidene)cyclohexan-1-one-2-(pyrrolidin-1-yl) ethyl)oxime

ESI-MS m/z: 523.20. 1H NMR (500 MHz, CDCl₃) δ ppm 7.67 (d, J=7.6 Hz, 2H), 7.50 (dd, J=17.0, 8.0 Hz, 2H), 7.43 (s, 1H), 7.35 (dt, J=16.4, 7.8 Hz, 4H), 7.05 (s, 1H), 4.38 (t, J=6.0 Hz, 2H), 2.89 (t, J=6.0 Hz, 2H), 2.63 (s, 4H), 2.47 (t, J=6.1 Hz, 2H), 2.39-2.32 (m, 2H), 1.82-1.72 (m, 4H), 1.60-1.52 (m, 2H).

### Example 6

2,6-(E,E)-(2-(trifluoromethyl)benzylidene)cyclohexan-1-one-3-(pyrrolidin-1-yl)propyl oxime

### ESI-MS m/z: 537.20.

### Example 7

### 2,6-(E,E)-(2-(trifluoromethyl)benzylidene)cyclohexan-1-one-2-(morpholinoethyl)oxime

ESI-MS m/z: 539.30. 1H NMR (500 MHz, CDCl₃) δ 7.60 (d, J=7.2 Hz, 2H), 7.42 (dd, J=16.3, 8.0 Hz, 2H), 7.34 (s, 1H), 7.30 (t, J=7.5 Hz, 2H), 7.22 (dd, J=20.1, 12.5 Hz, 2H), 6.96 (s, 1H), 4.31 (t, J=5.5 Hz, 2H), 3.67-3.56 (m, 4H), 2.72 (t, J=5.1 Hz, 2H), 2.51 (s, 4H), 2.39 (t, J=6.0 Hz, 2H), 2.32-2.23 (m, 2H), 1.48 (dd, J=12.3, 6.1 Hz, 2H).

### Example 8

### 2,6-(E,E)-(2-(trifluoromethyl)benzylidene)cyclohexan-1-one-3-(morpholinopropyl)oxime ESI-MS m/z: 553.20.

### Example 9

2,6-(E,E)-(2-(trifluoromethyl)benzylidene)cyclohexanone oxime

ESI-MS m/z: 426.10. 1H NMR (400 MHz, CDCl₃) δ ppm 7.66 (t, J=8.5 Hz, 2H), 7.54-7.46 (m, 2H), 7.44 (t, J=7.6 Hz, 1H), 7.41-7.32 (m, 3H), 7.29 (dd, J=14.9, 7.5 Hz, 2H), 2.49-2.33 (m, 4H), 1.63-1.52 (m, 2H).

### Example 10

2,6-(E,E)-(2-(trifluoromethyl)benzylidene)cyclohexan-1-one isopropyl oxime ESI-MS m/z: 468.20.

### Example 11

2,6-(E,E)-(2-(trifluoromethyl)benzylidene)cyclohexan-1-one-(2-methoxyethyl)oxime

ESI-MS m/z: 484.10. 1H NMR (400 MHz, CDCl₃) δ ppm 7.68 (d, J=7.9 Hz, 2H), 7.51 (t, J=6.7 Hz, 3H), 7.37 (dd, J=8.0, 3.7 Hz, 2H), 7.34 (d, J=4.9 Hz, 2H), 7.06 (s, 1H), 4.39-4.34 (m, 2H), 3.75-3.70 (m, 2H), 3.41 (s, 3H), 2.48 (t, J=5.8 Hz, 2H), 2.38-2.33 (m, 2H), 1.58 (dt, J=12.6, 6.3 Hz, 2H).

### Example 12

2,6-(E,E)-(2-(trifluoromethyl)benzylidene)cyclohexan-1-one-(2,2-dimethoxyethyl)oxime ESI-MS m/z: 514.20.

### Example 13

2,6-(E,E)-(2-(trifluoromethyl)benzylidene)cyclohexan-1-one cyclopentylmethyl oxime

ESI-MS m/z: 508.30. 1H NMR (400 MHz, CDCl₃) δ ppm 7.68 (dd, J=7.6, 3.5 Hz, 2H), 7.50 (dd, J=16.6, 7.3 Hz, 3H), 7.35 (dt, J=13.6, 6.9 Hz, 4H), 7.06 (s, 1H), 4.12 (d, J=7.2 Hz, 2H), 2.48 (t, J=6.0 Hz, 2H), 2.37-2.33 (m, 2H), 1.81-1.74 (m, 2H), 1.67-1.49 (m, 8H), 1.35 (d, J=7.1 Hz, 1H).

### Example 14

2,6-(E,E)-(2-(trifluoromethyl)benzylidene)cyclohexan-1-one cyclohexylmethyl oxime ESI-MS m/z: 522.30.

### Example 15

### 2,6-(E,E)-(2-(trifluoromethyl)benzylidene)cyclohexan-1-one-4-(methylbenzyl)oxime

ESI-MS m/z: 530.70. 1H NMR (400 MHz, CDCl₃) δ ppm 7.71-7.65 (m, 2H), 7.53-7.47 (m, 3H), 7.35 (dt, J=17.9, 6.8 Hz, 6H), 7.17 (d, J=7.8 Hz, 2H), 7.08 (s, 1H), 5.25 (s, 2H), 2.48 (t, J=5.9 Hz, 2H), 2.37 (d, J=7.8 Hz, 5H), 1.62-1.54 (m, 2H).

### Example 16

2,6-(E,E)-(2-(trifluoromethyl)benzylidene)cyclohexan-1-one-3-methoxybenzyl oxime ESI-MS m/z: 546.20.

### Example 17

2,6-(E,E)-(2-(trifluoromethyl)benzylidene)cyclohexan-1-one-3-chlorobenzyl oxime

ESI-MS m/z: 550.70. 1H NMR (400 MHz, CDCl₃) δ ppm 7.68 (d, J=7.8 Hz, 2H), 7.51 (dd, J=15.4, 7.5 Hz, 3H), 7.42 (s, 1H), 7.40-7.32 (m, 4H), 7.31-7.26 (m, 3H), 7.06 (s, 1H), 5.24 (s, 2H), 2.48 (t, J=5.9 Hz, 2H), 2.41-2.35 (m, 2H), 1.64-1.55 (m, 2H).

### Example 18

2,6-(E,E)-(2-(trifluoromethyl)benzylidene)cyclohexan-1-one-3-fluorobenzyl oxime ESI-MS m/z: 534.10.

### Example 19

2,6-(E,E)-(2-(trifluoromethyl)benzylidene)cyclohexan-1-one-4-fluorobenzyl oxime

ESI-MS m/z: 534.10. 1H NMR (400 MHz, CDCl₃) δ ppm 7.68 (dd, J=7.7, 4.6 Hz, 2H), 7.53-7.47 (m, 3H), 7.43-7.38 (m, 3H), 7.36 (d, J=6.6 Hz, 2H), 7.34-7.30 (m, 1H), 7.08-7.01 (m, 3H), 5.23 (s, 2H), 2.49 (t, J=5.8 Hz, 2H), 2.37 (t, J=5.9 Hz, 2H), 1.63-1.55 (m, 2H).

### Example 20

2,6-(E,E)-(2-(trifluoromethyl)benzylidene)cyclohexan-1-one-2,6-dichlorobenzyl oxime ESI-MS m/z: 586.10.

### Example 21

2,6-(E,E)-(2-(trifluoromethyl)benzylidene)cyclohexan-1-ketophenethyl oxime

ESI-MS m/z: 530.10. 1H NMR (400 MHz, CDCl₃) δ ppm 7.69 (d, J=7.7 Hz, 3H), 7.51 (q, J=7.5 Hz, 2H), 7.38 (dd, J=14.5, 5.7 Hz, 4H), 7.30 (d, J=8.8 Hz, 2H), 7.24 (dd, J=8.5, 2.5 Hz, 2H), 7.18 (ddd, J=8.5, 5.2, 2.8 Hz, 1H), 7.07 (s, 1H), 4.45 (t, J=7.0 Hz, 2H), 3.09 (q, J=6.6 Hz, 2H), 2.48 (t, J=5.9 Hz, 2H), 2.36 (t, J=6.0 Hz, 2H), 1.61-1.53 (m, 2H).

### Example 22

### 2,6-(E,E)-(2-(trifluoromethyl)benzylidene)cyclohexan-1-one-3-(methylphenylethyl)oxime ESI-MS m/z: 544.70.

### Example 23

### 2,6-(E,E)-(2-(trifluoromethyl)benzylidene)cyclohexan-1-one-2-(fluorophenethyl)oxime

ESI-MS m/z: 548.30. 1H NMR (400 MHz, CDCl₃) δ ppm 7.74-7.68 (m, 2H), 7.54 (dd, J=12.2, 7.3 Hz, 2H), 7.44-7.35 (m, 3H), 7.28 (dt, J=7.5, 5.3 Hz, 4H), 7.22-7.15 (m, 1H), 7.07 (s, 1H), 7.05-6.97 (m, 2H), 4.48 (t, J=6.7 Hz, 2H), 3.14 (t, J=6.6 Hz, 2H), 2.50 (t, J=5.9 Hz, 2H), 2.38 (t, J=6.0 Hz, 2H), 1.59 (dt, J=12.5, 6.3 Hz, 2H).

### Example 24

### 2,6-(E,E)-(2-(trifluoromethyl)benzylidene)cyclohexan-1-one-3-(fluorophenethyl)oxime

ESI-MS m/z: 548.30.

### Example 25

### 2,6-(E,E)-(2-(trifluoromethyl)benzylidene)cyclohexan-1-one-4-(fluorophenethyl)oxime

ESI-MS m/z: 548.30. 1H NMR (400 MHz, CDCl₃) δ ppm 7.69 (d, J=7.8 Hz, 2H), 7.51 (dd, J=16.1, 8.0 Hz, 2H), 7.42-7.33 (m, 3H), 7.33-7.27 (m, 2H), 7.23-7.17 (m, 2H), 7.06 (s, 1H), 7.00-6.88 (m, 2H), 4.41 (t, J=6.8 Hz, 2H), 3.04 (t, J=6.7 Hz, 2H), 2.48 (t, J=5.9 Hz, 2H), 2.36 (t, J=6.0 Hz, 2H), 1.62-1.53 (m, 2H).

### Example 26

2,6-(E,E)-(2-(trifluoromethyl)benzylidene)cyclohexan-1-one-((tetrahydrofuran-2-yl) methyl)oxime

ESI-MS m/z: 510.20.

### Example 27

2,6-(E,E)-(2-(trifluoromethyl)benzylidene)cyclohexan-1-one-((1,3-dioxolan-2-yl) methyl)oxime

ESI-MS m/z: 512.30. 1H NMR (400 MHz, CDCl₃) δ ppm 7.67 (d, J=7.8 Hz, 2H), 7.50 (dd, J=14.4, 7.1 Hz, 3H), 7.35 (ddd, J=10.4, 7.9, 3.4 Hz, 4H), 7.06 (s, 1H), 5.28 (t, J=3.9 Hz, 1H), 4.28 (d, J=3.9 Hz, 2H), 4.02-3.97 (m, 2H), 3.94-3.89 (m, 2H), 2.46 (t, J=5.9 Hz, 2H), 2.36 (t, J=6.0 Hz, 2H), 1.56 (dt, J=12.6, 6.3 Hz, 2H).

### Example 28

2,6-(E,E)-(2-(trifluoromethyl)benzylidene)cyclohexan-1-one-(tetrahydro-2H-pyran-4-yl) methyl oxime

ESI-MS m/z: 524.30.

### Example 29

2,6-(E,E)-(2-(trifluoromethyl)benzylidene)cyclohexan-1-one-2-(1,3-dioxolan-2-yl) ethyl)oxime

ESI-MS m/z: 526.30. 1H NMR (400 MHz, CDCl₃) δ ppm 7.67 (d, J=7.8 Hz, 2H), 7.50 (dd, J=15.1, 7.8 Hz, 3H), 7.35 (dt, J=13.3, 7.1 Hz, 4H), 7.05 (s, 1H), 5.05 (t, J=5.0 Hz, 1H), 4.37 (t, J=6.5 Hz, 2H), 4.02-3.92 (m, 2H), 3.90-3.82 (m, 2H), 2.47 (t, J=5.8 Hz, 2H), 2.38-2.31 (m, 2H), 2.16-2.09 (m, 2H), 1.61-1.53 (m, 2H).

### Example 30

2,6-(E,E)-(2-(trifluoromethyl)benzylidene)cyclohexan-1-one-(3-(4-methylpiperazin-1-yl) propyl)oxime

ESI-MS m/z: 566.40.

### Example 31

2,6-(E,E)-(2-(trifluoromethyl)benzylidene)cyclohexan-1-one-(3-(piperidin-1-yl)propyl) oxime

ESI-MS m/z: 551.30. 1H NMR (400 MHz, CDCl₃) δ ppm 7.69-7.63 (m, 2H), 7.49 (dd, J=15.6, 7.9 Hz, 3H), 7.38-7.29 (m, 4H), 7.04 (s, 1H), 4.25 (t, J=6.3 Hz, 2H), 2.45 (dd, J=13.9, 6.5 Hz, 4H), 2.36 (dd, J=13.2, 6.7 Hz, 6H), 1.99-1.90 (m, 2H), 1.61-1.52 (m, 6H), 1.41 (d, J=4.8 Hz, 2H).

### Example 32

2,6-(E,E)-(benzylidene)cyclohexan-1-one propyl oxime

ESI-MS m/z: 332.09.

### Example 33

2,6-(E,E)-(2-(fluoro)benzylidene)cyclohexan-1-one isopropyl oxime

ESI-MS m/z: 368.07. 1H NMR (400 MHz, CDCl₃) δ ppm 7.92 (s, 2H), 7.37-7.39 (m, 2H), 7.18-7.21 (m, 4H), 7.13-7.17 (m, 2H), 4.48 (t, J=6.2 Hz, 1H), 2.48 (dd, J=9.1, 3.6 Hz, 2H), 2.37-2.31 (m, 2H), 1.62-1.58 (m, 2H), 1.33 (d, J=6.2 Hz, 6H).

### Example 34

2,6-(E,E)-(4-(bromo)benzylidene)cyclohexan-1-one isopropyl oxime

ESI-MS m/z: 487.91.

### Example 35

2,6-(E,E)-(4-(methoxy)benzylidene)cyclohexan-1-one-(2-methoxyethyl)oxime

ESI-MS m/z: 408.01.1H NMR (400 MHz, CDCl₃) δ ppm 7.79 (s, 2H), 7.29 (d, 4H), 6.95 (d, 4H), 4.39-4.34 (m, 2H), 3.75-3.70 (m, 2H), 3.41 (s, 3H), 2.48 (t, J=5.8 Hz, 2H), 2.38-2.33 (m, 2H), 1.58 (dt, J=12.6, 6.3 Hz, 2H).

### Example 36

2,6-(E,E)-(4-(methoxy)benzylidene)cyclohexan-1-one cyclopentylmethyl oxime

ESI-MS m/z: 432.05.

### Example 37

2,6-(E,E)-(4-(hydroxy)benzylidene)cyclohexan-1-one cyclopentylmethyl oxime

ESI-MS m/z: 404.11. 1H NMR (400 MHz, CDCl₃) δ ppm 8.93 (s, 2H), 7.65 (s, 2H), 7.31 (d, J=8.4 Hz, 4H), 6.94 (d, J=8.4 Hz, 4H), 4.12 (d, J=7.2 Hz, 2H), 2.48 (t, J=6.0 Hz, 2H), 2.37-2.33 (m, 2H), 1.81-1.74 (m, 2H), 1.67-1.49 (m, 8H), 1.35 (d, J=7.1 Hz, 1H).

### Example 38

### 2,6-(E,E)-(4-(hydroxy)benzylidene)cyclohexan-1-one-2-(morpholinoethyl)oxime

ESI-MS m/z: 435.34.

### Example 39

2,6-(E,E)-(3,4-(dichloro) benzylidene)cyclohexan-1-one-2-(morpholinoethyl)oxime

ESI-MS m/z: 539.07. 1H NMR (400 MHz, CDCl₃) δ ppm 7.67 (s, 2H), 7.50 (d, J=8.4 Hz, 2H), 7.46 (s, 2H), 7.21 (d, J=7.8 Hz, 2H), 4.31 (t, J=5.5 Hz, 2H), 3.67-3.56 (m, 4H), 2.72 (t, J=5.1 Hz, 2H), 2.51 (s, 4H), 2.39 (t, J=6.0 Hz, 2H), 2.32-2.23 (m, 2H), 1.48 (dd, J=12.3, 6.1 Hz, 2H).

### Example 40

2,6-(E,E)-(2-(hydroxy) benzylidene)cyclohexan-1-one propyl oxime

ESI-MS m/z: 364.18.

### Pharmacological Studies of Product of The Present Invention

Dose-Effect Relationship of Compounds of Examples in Inhibiting Release of Inflammatory Factors from Macrophages Stimulated by LPS

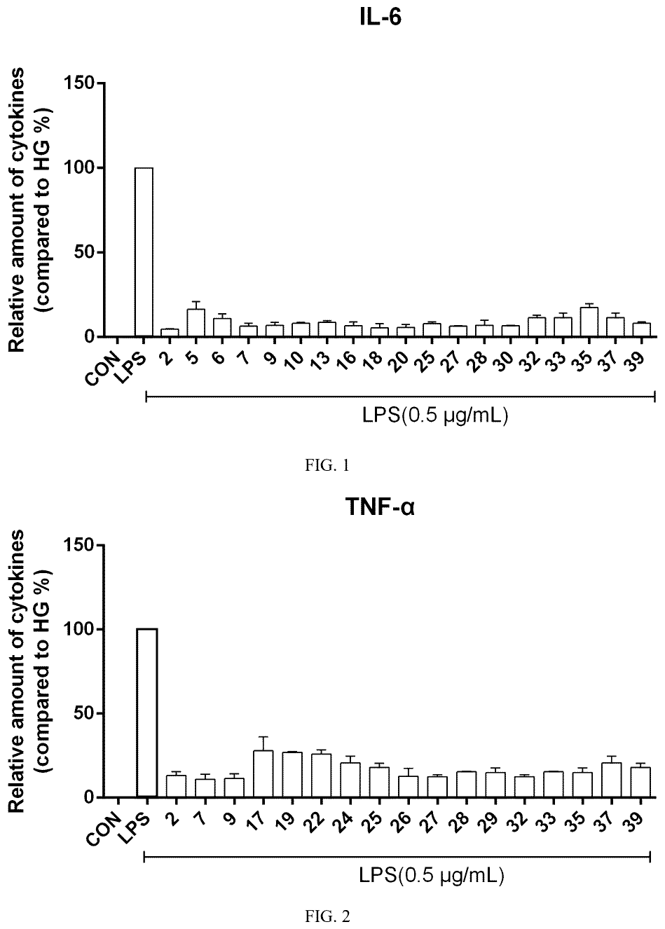

The present invention tested the dose-effect relationship of some of the compounds of Examples in inhibiting the release of IL-6 and TNF-α from J774A.1 macrophages stimulated by LPS. The specific method is as follows: 1.2×10⁶ primary macrophages were cultured by DMEM culture solution at 37° C., the culture solution was renewed after 24 h, the tested example compound (at a final concentration of 10 μM) was added for pretreatment for 2 h and then continuously treated by 0.5 μg/mL of LPS for 22 h, the culture solution was collected, and the content of IL-6 and TNF-α was detected by ELISA method. The total protein concentration was measured by collecting cells, the ELISA results were divided by the corresponding total protein concentration and calibrated by the IL-6 and TNF-α content of LPS control group as 100, and the average and error values were calculated. The experimental results are shown in FIG. 1 and FIG. 2. It can be seen from FIG. 1 that the tested example compounds 2, 5, 6, 7, 9, 10, 13, 16, 18, 20, 25, 27, 28, 30, 32, 33, 35, 37, 39 have a significant inhibitory effect on the release of IL-6. It can be seen from FIG. 2 that the tested example compounds 2, 7, 9, 17, 19, 22, 24, 25, 26, 27, 28, 29, 32, 33, 35, 37, 39 have a significant inhibitory effect on the release of TNF-α. The compounds of Examples of the present invention all have the skeleton structure of the compound of formula I, and also incorporate fragments with an excellent anti-inflammatory activity, indicating that the compound of formula I of the present invention has an excellent anti-inflammatory activity.

### Protective Effect of Compounds of Examples on Liver Inflammation in Obese Mice

A suspension of 0.5% sodium carboxymethylcellulose and the compounds of Examples was prepared for intragastric administration. The blank control group and the high-fat diet obesity model group were administrated with equal doses of solvent (0.5% CMC-Na solution) intragastrically. The mice were first adaptively raised for 1 week, and when the mice were 8 weeks old, they were randomly divided into 2 groups (A: Con, B: HFD). Group A was fed with normal mouse feed, and mice in group B were fed with 60% high-fat feed for 16 weeks. After the model was successfully established at 12 weeks, the mice in group B were randomly divided into three groups (i: HFD, ii: HFD+Example 9, 12 mg/kg, iii: HFD+Example 2, 10 mg/kg). The body weight of the mice was measured and recorded every week, and groups ii and iii were treated with the compounds of Examples by intragastric gavage every two days for 8 weeks. After 24 weeks, the mice were anesthetized and sacrificed, and blood samples were collected and centrifuged to collect serum for various serum biochemical tests; the liver was removed, and the liver tissue was cut and embedded (frozen and paraffin) for immunohistochemistry and other analyses. The remaining liver tissues were snap frozen in liquid nitrogen and stored in a −80° C. freezer.

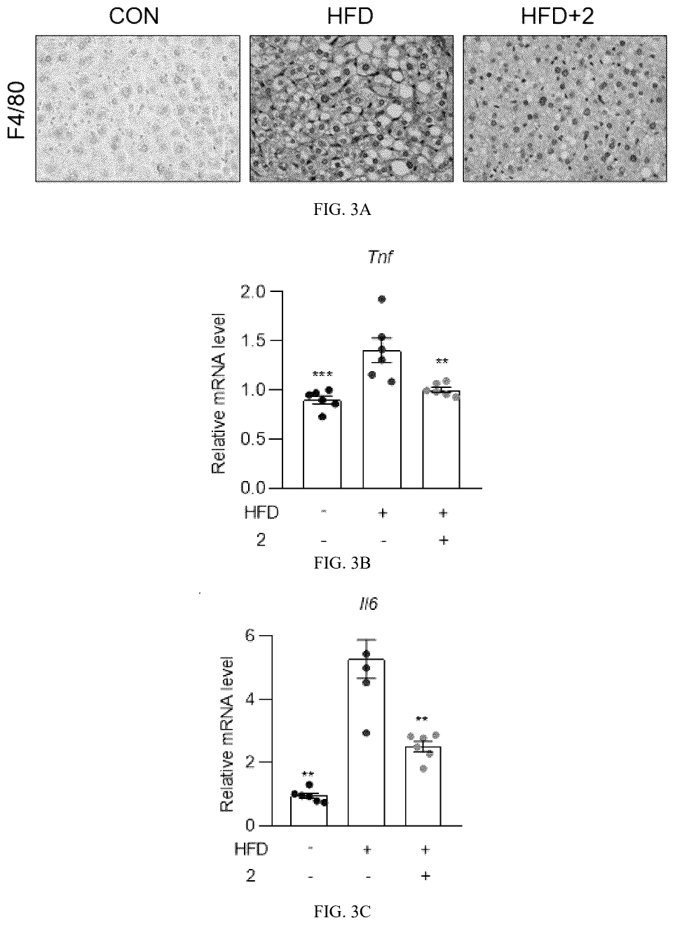

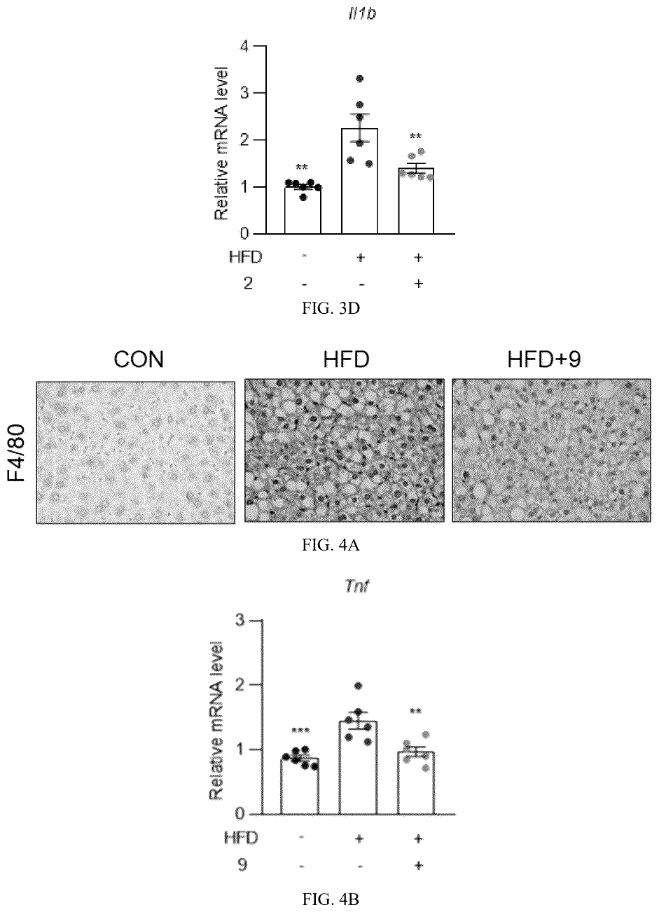

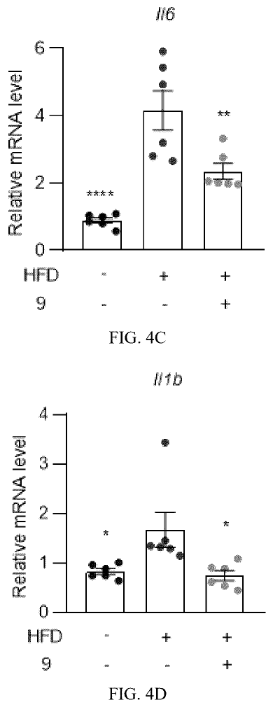

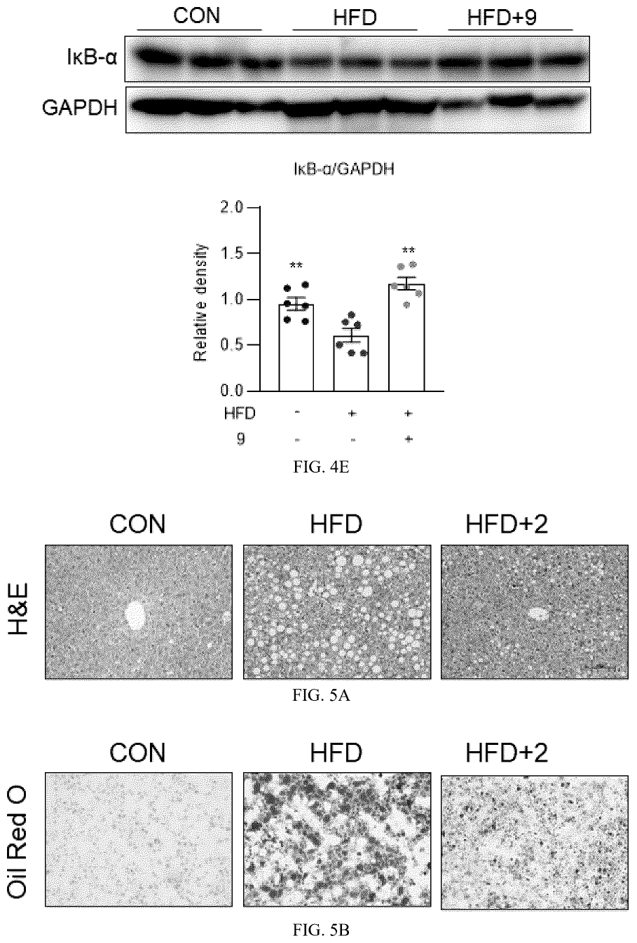

Taking the compounds of Examples 2 and 9 as examples, the experimental results are shown in FIGS. 3A-3D and FIGS. 4A-4E, where 2 and 9 represent the compounds of Example 2 and Example 9, respectively. Inflammation was assessed by examining macrophage infiltration in the liver using the macrophage-specific marker F4/80 staining. The results in FIGS. 3A-3D and FIGS. 4A-4E show that macrophage infiltration in the liver of mice fed a high-fat diet significantly increased, and macrophage infiltration decreased after treatment with the compounds of Examples 2 and 9. In addition, the expression of TNF-α, IL-6 and IL-1β genes in liver tissue was detected. The results show that non-alcoholic fatty liver disease causes a significant upregulation of TNF-α, IL-6 and IL-1β genes in mouse liver tissue, which is significantly alleviated after administration of the compounds of Examples 2 and 9. Western blot analysis proves that the compound of Example 9 can significantly inhibit the degradation of IκB-α in the liver caused by high-fat diet feeding, indicating that the compound of the present invention has a protective effect on liver inflammation in obese mice.

### Protective Effect of Compounds in Examples on Liver Lipid Metabolism of Obese Mice

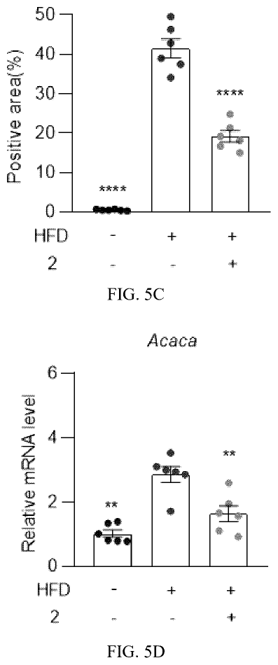

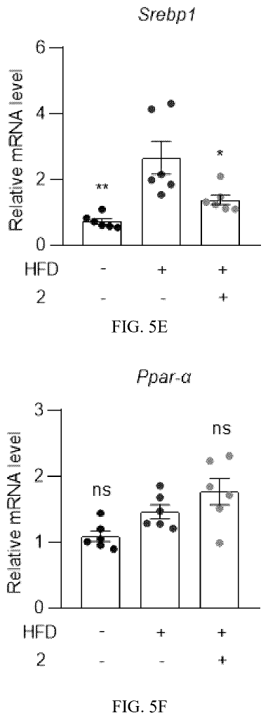

The liver lipid metabolism of obese mice is taken as an example. The experimental results are shown in FIGS. 5A-5F and FIGS. 6A-6F. It can be seen from FIGS. 5A-5F and FIGS. 6A-6F that, by detecting the levels of serum biochemical indicators such as triglycerides, low-density lipoprotein cholesterol and total cholesterol in mice, dyslipidemia caused by high-fat diet feeding was significantly alleviated after administration of the compounds of Examples 2 and 9. The results of hematoxylin&eosin (H&E) staining of the liver show that the lipid accumulation in the liver caused by a high-fat diet is significantly alleviated after treatment with the compounds of Examples 2 and 9. The frozen sections of the liver were stained with oil red, and after analysis and quantification, it is found that the treatment with the compounds of Examples 2 and 9 significantly alleviates the lipid accumulation in the liver. It can be clearly seen from the above test results that the compound having the structure of formula I of the present invention has a protective effect on the liver lipid metabolism of obese mice.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A 2,6-dibenzylidenecyclohexanone oxime compound, wherein the 2,6-dibenzylidenecyclohexanone oxime compound is a compound with a structure of a formula I or a pharmaceutically acceptable salt thereof: 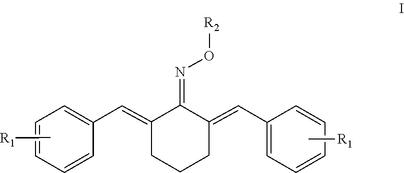
in the formula I, R₁ is independently one of fluorine, chlorine, trifluoromethyl, methoxy, or hydroxyl; and
R₂ is independently one of hydrogen,

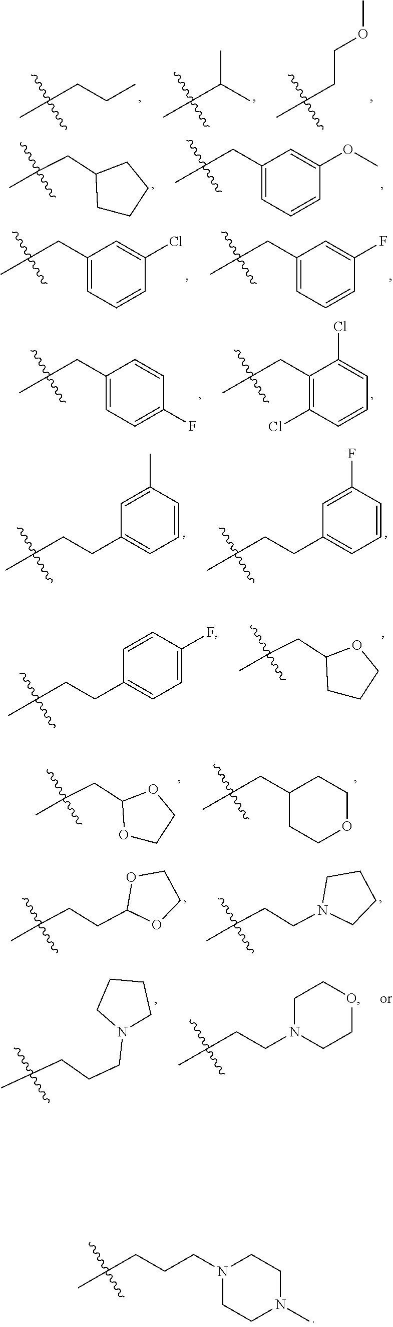

2. The 2,6-dibenzylidenecyclohexanone oxime compound according to claim 1, wherein the 2,6-dibenzylidenecyclohexanone oxime compound is the following compound or a pharmaceutically acceptable salt thereof:
2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(pyrrolidin-1-yl)ethyl oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one isopropyl oxime;
2,6-(E,E)-(2-(trifluoromethyl)benzylidene)cyclohexanone oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one-((tetrahydrofuran-2-yl) methyl)oxime;
2,6-(E,E)-(2-(trifluoromethyl)benzylidene)cyclohexan-1-one-((1,3-dioxolan-2-yl) methyl)oxime;
2,6-(E,E)-(2-(trifluoromethyl)benzylidene)cyclohexan-1-one-(tetrahydro-2H-pyran-4-yl) methyl oxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(fluoro)benzylidene)cyclohexan-1-one isopropyl oxime;
2,6-(E,E)-(4-(methoxy)benzylidene)cyclohexan-1-one-(2-methoxyethyl)oxime;
2,6-(E,E)-(4-(hydroxy)benzylidene)cyclohexan-1-one cyclopentylmethyl oxime; and
2,6-(E,E)-(3,4-(dichloro) benzylidene)cyclohexan-1-one-2-(morpholinoethyl)oxime.

3. A method for preparing a medicament for a treatment of an inflammation, comprising using the 2,6-dibenzylidenecyclohexanone oxime compound according to claim 1.

4. A method for preparing a medicament for a treatment of fatty liver, comprising using the 2,6-dibenzylidenecyclohexanone oxime compound according to claim 1, wherein the 2,6-dibenzylidenecyclohexanone oxime compound is 2,6-(E,E)-(2-(trifluoromethyl) benzylidene)cyclohexan-1-one propyl oxime or 2,6-(E,E)-(2-(trifluoromethyl) benzylidene)cyclohexanone oxime.

5. A pharmaceutical preparation, comprising an active ingredient and a pharmaceutic adjuvant; wherein the active ingredient comprises the 2,6-dibenzylidenecyclohexanone oxime compound according to claim 1; and the pharmaceutical preparation is one of an injection, a tablet, a capsule, an aerosol, a suppository, a film, a dropping pill, an ointment, a controlled-release agent, a sustained-release agent, or a nano-preparation.

6. The method for preparing the medicament for the treatment of the inflammation according to claim 3, wherein the 2,6-dibenzylidenecyclohexanone oxime compound is the following compound or a pharmaceutically acceptable salt thereof:
2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(pyrrolidin-1-yl)ethyl oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one isopropyl oxime;
2,6-(E,E)-(2-(trifluoromethyl)benzylidene)cyclohexanone oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one- ((tetrahydrofuran-2-yl) methyl)oxime;
2,6-(E,E)-(2-(trifluoromethyl)benzylidene)cyclohexan-1-one- ((1,3-dioxolan-2-yl) methyl)oxime;
2,6-(E,E)-(2-(trifluoromethyl)benzylidene)cyclohexan-1-one-(tetrahydro-2H-pyran-4-yl) methyl oxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(fluoro)benzylidene)cyclohexan-1-one isopropyl oxime;
2,6-(E,E)-(4-(methoxy)benzylidene)cyclohexan-1-one-(2-methoxyethyl)oxime;
2,6-(E,E)-(4-(hydroxy)benzylidene)cyclohexan-1-one cyclopentylmethyl oxime; and
2,6-(E,E)-(3,4-(dichloro)benzylidene)cyclohexan-1-one-2-(morpholinoethyl)oxime.

7. The method for preparing the medicament for the treatment of the fatty liver according to claim 4, wherein the 2,6-dibenzylidenecyclohexanone oxime compound is the following compound or a pharmaceutically acceptable salt thereof:
the 2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(pyrrolidin-1-yl)ethyl oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one isopropyl oxime;
the 2,6-(E,E)-(2-(trifluoromethyl)benzylidene)cyclohexanone oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one- ((tetrahydrofuran-2-yl) methyl)oxime;
2,6-(E,E)-(2-(trifluoromethyl)benzylidene)cyclohexan-1-one- ((1,3-dioxolan-2-yl) methyl)oxime;
2,6-(E,E)-(2-(trifluoromethyl)benzylidene)cyclohexan-1-one-(tetrahydro-2H-pyran-4-yl) methyl oxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(fluoro)benzylidene)cyclohexan-1-one isopropyl oxime;
2,6-(E,E)-(4-(methoxy)benzylidene)cyclohexan-1-one-(2-methoxyethyl)oxime;
2,6-(E,E)-(4-(hydroxy)benzylidene)cyclohexan-1-one cyclopentylmethyl oxime; and
2,6-(E,E)-(3,4-(dichloro)benzylidene)cyclohexan-1-one-2-(morpholinoethyl)oxime.

8. The pharmaceutical preparation according to claim 5, wherein the 2,6-dibenzylidenecyclohexanone oxime compound is the following compound or a pharmaceutically acceptable salt thereof:
2,6-(E,E)-(2-(trifluoromethyl)benzylidene)cyclohexan-1-one propyl oxime;
2,6-(E,E)-(2-(trifluoromethyl)benzylidene)cyclohexan-1-one-2-(pyrrolidin-1-yl)ethyl oxime;
2,6-(E,E)-(2-(trifluoromethyl)benzylidene)cyclohexan-1-one-3-(pyrrolidin-1-yl)propyl oxime;
2,6-(E,E)-(2-(trifluoromethyl)benzylidene)cyclohexan-1-one-2-(morpholinoethyl)oxime;
2,6-(E,E)-(2-(trifluoromethyl)benzylidene)cyclohexan-1-one isopropyl oxime;
2,6-(E,E)-(2-(trifluoromethyl)benzylidene)cyclohexanone oxime;
2,6-(E,E)-(2-(trifluoromethyl)benzylidene)cyclohexan-1-one cyclopentylmethyl oxime;
2,6-(E,E)-(2-(trifluoromethyl)benzylidene)cyclohexan-1-one-3-methoxybenzyl oxime;
2,6-(E,E)-(2-(trifluoromethyl)benzylidene)cyclohexan-1-one-3-chlorobenzyl oxime;
2,6-(E,E)-(2-(trifluoromethyl)benzylidene)cyclohexan-1-one-3-fluorobenzyl oxime;
2,6-(E,E)-(2-(trifluoromethyl)benzylidene)cyclohexan-1-one-4-fluorobenzyl oxime;
2,6-(E,E)-(2-(trifluoromethyl)benzylidene)cyclohexan-1-one-2,6-dichlorobenzyl oxime;
2,6-(E,E)-(2-(trifluoromethyl)benzylidene)cyclohexan-1-one-3-(methylphenylethyl)oxime;
2,6-(E,E)-(2-(trifluoromethyl)benzylidene)cyclohexan-1-one-3-(fluorophenethyl)oxime;
2,6-(E,E)-(2-(trifluoromethyl)benzylidene)cyclohexan-1-one-4-(fluorophenethyl)oxime;
2,6-(E,E)-(2-(trifluoromethyl)benzylidene)cyclohexan-1-one-((tetrahydrofuran-2-yl) methyl)oxime;
2,6-(E,E)-(2-(trifluoromethyl)benzylidene)cyclohexan-1-one-((1,3-dioxolan-2-yl) methyl)oxime;
2,6-(E,E)-(2-(trifluoromethyl)benzylidene)cyclohexan-1-one-(tetrahydro-2H-pyran-4-yl) methyl oxime;
2,6-(E,E)-(2-(trifluoromethyl)benzylidene)cyclohexan-1-one-2-(1,3-dioxolan-2-yl) ethyl)oxime;
2,6-(E,E)-(2-(trifluoromethyl)benzylidene)cyclohexan-1-one-(3-(4-methylpiperazin-1-yl) propyl)oxime;
2,6-(E,E)-(2-(fluoro) benzylidene)cyclohexan-1-one isopropyl oxime;
2,6-(E,E)-(4-(methoxy) benzylidene)cyclohexan-1-one-(2-methoxyethyl)oxime;
2,6-(E,E)-(4-(hydroxy) benzylidene)cyclohexan-1-one cyclopentylmethyl oxime; and
2,6-(E,E)-(3,4-(dichloro) benzylidene)cyclohexan-1-one-2-(morpholinoethyl)oxime.
